(19) United States
(12) Patent Application Publication
(10) Pub. No.: US 2002/0192418 A1
(43) Pub. Date: Dec. 19, 2002

(54) Method of applying an optical security coating to a substrate

(76) Inventors: Daniel Lieberman Zadjman, Mexico (MX); Ramon Bautista Perez-Salazar, Mexico (MX)

Correspondence Address: BROWN, MARTIN, HALLER & MCCLAIN LLP, 1660 UNION STREET, SAN DIEGO, CA, 92101-2926, US

(21) Appl. No.: 10/219,193

(22) Filed: Aug. 14, 2002

Related U.S. Application Data
(62) Division of application No. 09/589,950, filed on Jun. 8, 2000, now Pat. No. 6,443,494.

(51) Int. Cl. A61F 13/02

(52) U.S. Cl. 428/42.1; 428/101

(57) ABSTRACT

This invention describes a removable optical security film of a type which includes a substrate with printed information and a first film which avoids the permanent adhesion of the optical security film to the substrate, whereby said optical film includes the temporary application of a removable material with adhesive characteristics on the substrate on which it is applied, and the engraving of optical elements on said removable material. In addition to authenticating the information printed underneath and avoiding the reprocessing of objects and prints, the optical security film also prevents the duplication of these objects and prints by conventional methods. In another embodiment of this invention, when the optical security film acts partially, the print and the non-removed parts of the film are the elements that authenticate the corresponding information.

### FIELD OF THE INVENTION

This invention concerns the protection of a print and/or object by applying a removable film to a surface. More specifically, this invention concerns a removable optical security film and its application, to totally or partially hide confidential information previously printed on a surface and to authenticate said information.

### BACKGROUND OF THE INVENTION

Currently, there exists in the market a series of printed documents such as calling cards, instant lottery tickets, promotions, licenses, event tickets and similar items, which contain information, activation codes, prizes, etc. hidden by means of a removable material. The purpose of the removable material is to ensure the confidentiality of the code or message printed under the material before it is acquired by a final consumer. However, a problem faced by this type of application of removable material on products is the possibility that, after the removable material is removed from the surface and the hidden information is used, the document is then discarded by the user. However, the discarded document, which no longer has any commercial value, can be picked up and reused illegally, by reapplying the removable material in order to sell it again; furthermore, the document may be printed by any conventional printing method to imitate the original, imitating both the print and the film.

To date, there is no security measure to easily show or identify the reprocessing or duplication of documents already used, i.e. the authenticity of the documents, so users who purchase these reprocessed documents suffer an economic loss, and the manufacturers or issuers suffer damage to their image, in addition to economic losses.

The current technique involves the application of a removable material to a smooth surface by conventional printing methods such as flexography, serigraphy, offset, rotogravure, hot stamping, etc. However, said removable film does not have security measures or elements to identify reprocessed or duplicated documents, so any printer may easily reapply the film and/or copy the print with the film by conventional printing methods.

Consequently, there is still a need to provide a security measure or removable optical film identifying undue use and reprocessing or duplication, which security measure has a correct balance between adhesion strength and removal strength (friction) of the security measure, in order to show the information it hides.

### OBJECTIVES OF THE INVENTION

Taking into account the shortcomings of the above technique, one of this invention's objectives is to provide an optical security film that is easy to apply but highly efficient for the authentication and validation of documents containing confidential information.

Another objective of this invention is to provide an optical security film that may be applied by any traditional or non-traditional method.

Yet another objective of this invention is to provide an optical security film with optical characteristics that cannot be duplicated by conventional printing methods.

One more objective of this invention is to provide an optical security film that has the correct balance between adhesion strength and removal strength.

The above objectives, as well as other objectives and advantages of this invention, are achieved by providing a removable optical security film to totally or partially cover a smooth surface, in order to authenticate and validate the confidential information previously printed on said surface. In addition to the optical characteristics, this invention's optical security film also has a correct balance between adhesion strength and removal strength (friction), which gives it special additional characteristics as a security element preventing the reprocessing of documents already used. Said optical film may include any type of optical elements that give consumers the security that the print and/or object has not been reused or duplicated, and therefore that the hidden information has not been seen or used by anybody else, thus assuring its confidentiality.

An additional characteristic of the removable optical security film is that it can be opaque or transparent, i.e. the purpose of the opaque optical film will be to hide the information, while the transparent optical film will validate the visible information since, by removing the film, the optical effect validating the information underneath will appear.

Finally, since the optical security film can be partially removed, this allows the printed information, together with the permanent parts of the film (after the film is removed), to give us the correct code to validate the printed information.

### DETAILED DESCRIPTION OF THE INVENTION

This invention involves an optical security film that can be applied by any of the printing methods known in the field. The optical security film can be applied by any method known to printing experts, such as flexography, serigraphy, offset, rotogravure, labeling, hot stamping, etc. However, this invention's removable film has optical characteristics that provide a security element to the document or surface to which it is applied. Furthermore, said removable film has specific characteristics that prevent the reuse or reprocessing of the substrate to which it is initially applied, giving the final user security that the information contained in the document has been kept confidential.

This invention's optical film may be totally or partially removed, showing that the print and/or object have already been used, thus offering an element by which the user can easily and fully identify the print and/or object as original.

As used herein, the term “optical film” refers to the microscopic engraving of any optical element on a film. The optical elements that may be engraved microscopically on a film can be, without limitation thereto, holograms, diffraction gradients, optically variable diffraction elements (OVD), dot matrix elements (at any resolution), computer-generated holograms, stereograms, hexelgrams, kinegrams, etc. This type of optical element generates optical reliefs.

It must be understood that the incorporation of the optical element or elements is not limited to the aforementioned interference patterns; it may be extrapolated to give the optical film optical elements generated in the volume of the film by the well-known system used to generate holograms and/or optical reflection elements.

The type of information protected with this invention's removable optical security film can be, for example, activation codes, prizes, codes, logos, photographs, numbers, etc. This confidential information is contained in printed documents such as calling cards, instant lottery tickets, promotions, licenses, collectible cards, event tickets and similar documents.

Generally speaking, this invention's removable optical film includes both the temporary application of a removable material with adhesive characteristics on the substrate to which it is applied and the engraving of optical elements on said removable material. In this sense, the correct balance will be achieved if the removable material and the optical elements resist the friction forces generated, for example, during packing, distribution, exhibition, etc., when handling the product to which it is applied; at the same time, it can easily be removed in order to see the information underneath the optical security film. Thus, excessive adherence strength of the removable material will cause the destruction of the base material (print) when trying to remove the film which contains the security measure to prevent reading, and a lack of adherence strength will make the removable material peel off when the product is handled, thus making the product unusable, because the hidden information would be exposed.

An additional characteristic of the removable optical security film is that it can be either opaque or transparent. In an embodiment of this invention, the opaque optical film has the purpose of hiding all the information found under the security film, while a transparent optical film will validate the visible information when, after removing the film, the optical effect validating the information underneath finally appears. In other words, the transparent optical security film will validate the information when it is under a transparent removable film with the same refraction index. By removing the transparent removable film, the optical security effect engraved underneath, on the other fixed, transparent film, will be activated, validating the information printed on the document or object.

### EXAMPLES

The following are illustrative examples, without limitation thereto, of some of the methods used to apply this invention's removable optical security film.

1. Hot Stamping Method

This method requires the substrate to which this invention's film will be applied to include a film which prevents the hot stamping from remaining permanently adhered to the substrate, making it adhere temporarily. For this purpose, as one example among many, the substrate may have a film of some type of lacquer containing, for example, silicone, which will prevent the permanent adhesion of the hot stamping. It is also necessary for the hot stamping to have adequate properties to adhere temporarily to said film.

Printing Station

The removable film may be applied to the substrate by flexography. This printing method by flexography allows a type of lacquer containing silicone (or another stripping material) to be impregnated by using anilox rollers on the substrate, precisely on the required area. Silicones or stripping materials are chemical products which, due to their physicochemical properties, prevent any substance or material from sticking to them. In this printing station, the lacquer is applied to the substrate, and then the object goes to a drying station, where the lacquer is dried on the substrate.

Anilox rollers have small cavities in which the lacquer is stored and from which it is transferred to a photo-polymeric stencil plate, which has the drawing of the area on which the application will be made. Thus, the stencil plate with said relief area takes ink from the anilox roller and transfers it to the substrate. After the lacquer is transferred to the substrate, the substrate is immediately moved to a drying station. These lacquers may be solvent-based, water-based or with ultraviolet curing.

Hot Stamping Station

At this station, the substrate is placed so that, by application of heat and pressure, an optical hot-stamping which has been engraved in this material is transferred. A key characteristic is that said optical hot-stamping must be transferred easily to the substrate and remain temporarily adhered to it. For this purpose, the proper formulation must be sought both in adhesives and in stripping agents, i.e. the correct balance between adhesion strength and removal strength, to cause the material to always work under the same pressure and temperature conditions.

For example, an optical hot-stamping under this invention may consist of a polyester “carrier” to which, first of all, stripping lacquer is applied to achieve the transfer. Then, over the first lacquer or wax, a second lacquer, which is optically engraved, is applied; on top of it, a metal coating is applied to give “optical effect” reflectiveness. Over the metal coating, an adhesive is applied, activated by heat, in order to achieve two things: to allow the structure laminated in this way to be removed from the polyester and for it to temporarily adhere to the substrate, thus forming this invention's removable optical security film.

After the application of this optical film, and due to its characteristics described above, it can be easily removed by scratching it, for example with a coin, etc. in the area where the security film was placed. This works in exactly the same way as scratching instant lottery tickets.

2. Ultraviolet Method

In this method, the substrate to which this invention's optical security film will be applied must also have a film that prevents the permanent adhesion of the removable film. For this purpose, a first film can be applied, such as X-type UV (ultraviolet) lacquer. This type of lacquer is formulated so that, after being cured, it provides increased resistance to friction. In addition, the chemical composition of said lacquer must be selected so as not to allow the permanent adhesion of the other type-Y lacquer cured over it.

The type-Y lacquer is designed so that, after being cured, it is easy to break by friction, so that it can be scratched without problems.

The application methodology basically consists of printing, engraving and curing.

Printing, Engraving and Curing Station

The removable film can be applied to the substrate by flexography. This flexographic printing method, which uses anilox rollers, allows the substrate to be impregnated precisely in the area where the application of a type-X UV lacquer is desired. Anilox rollers have small cavities in which the lacquer is stored and from which it is transferred to a photo-polymeric stencil plate, which has the drawing of the area on which the application will be made. Thus, the stencil plate with said relief area takes ink from the anilox roller and transfers it to the substrate. After the lacquer is transferred to the substrate, the substrate is immediately moved to a curing station with ultraviolet rays.

After the type-X lacquer is cured over the substrate, the substrate with the first film is transferred to a station where a second UV lacquer is applied, this being a type-Y lacquer. At this station, the type-Y lacquer is placed directly over the first UV lacquer, i.e. the type-X lacquer. After the application of the second lacquer, the substrate carrying both lacquers is moved to a semi-curing station, and from there it is transferred to an engraving station, where an engraving roller has the optical image that will be engraved over the semi-cured type-Y UV lacquer. After the optical image is engraved over the semi-cured lacquer on the substrate which contains the confidential information, it is sent to a final UV curing station in order to permanently affix the optical image on this type-Y lacquer.

Thus, by means of a film of type-Y lacquer over a film of type-X lacquer, it is very simple to scratch the film formed by the type-Y lacquer, due to its adhesive characteristics, which are lower than those of the type-X film; the latter, once it is cured, is highly resistant. The removable coating consisting of the type-Y lacquer (the coating with the optical image engraved) may be any color, including transparent. It is preferable to use black to be able to see the optical image more easily. This can also be done with a transparent lacquer, where the optical security effect is engraved in the type-X lacquer. In this type of method, it is not necessary to metallize the film which contains the engraved optical security element.

3. Cold Transfer Method

Under this method, the substrate on which this invention's removable optical film will be applied must include a lacquer film with, for example, silicone or UV (ultraviolet) lacquer, which prevents the removable film applied over it from remaining permanently adhered.

Printing Station

The removable film may be applied to the substrate by flexography. This printing method by flexography allows a type of lacquer containing, for example, silicone or another stripping material, or a type of ultraviolet lacquer, to be impregnated using anilox rollers on the substrate, precisely on the required area. At this printing station, the lacquer is applied to the substrate, and then the object goes to a drying or curing station (depending on the type of lacquer used), where the lacquer is dried or cured on the substrate, and then it is sent to a station where adhesive is applied. Anilox rollers have small cavities in which lacquer or ink is stored and from which it is transferred to a photo-polymeric stencil plate, which has the drawing of the area in which the application will be made. Thus, the stencil plate with said relief area takes ink from the anilox roller and transfers it to the substrate. After the lacquer is transferred to the substrate, the substrate is immediately moved to a drying or curing station. These lacquers may be solvent-based, water-based or with ultraviolet curing.

Adhesive Application Station

After the lacquer or ink coating is applied to the substrate, the substrate with said coating is transferred to a station where a special type of adhesive is applied. The adhesive used is adhered to the lacquer non-permanently but with sufficient adhering strength that, when it goes to the following station, the transfer material containing the optical image is transferred to a cold roller by applying pressure to the substrate and is fixed on it non-permanently, thanks to the adhesive.

Although special forms of embodiment of this invention have been illustrated and described, specialists in the field will clearly see that several other changes and modifications can be made without changing the spirit and the scope of the invention.-Consequently, the purpose of the enclosed claims is to protect any change or modification made within the scope of this invention.

1. Removable optical security film, of a type including a substrate which contains printed information and a first film which avoids permanent adhesion of the optical security film to the substrate, characterized both by the fact that it includes both the application of a removable material which temporarily adheres to the substrate to which it is applied, and by the engraving of optical elements on said removable material; in addition, the film may be eliminated if the optical security film has stripping agents which prevent it from being permanently adhered.

2. The removable optical security film under claim 1, also characterized by the fact that the removable material and the optical elements have the correct balance, which consists of resisting the frictional forces generated during packing, distribution, exhibition, when handling the product on which it is applied; at the same time, is easily removable to see the information contained under the optical security film.

3. The removable optical security film under claim 1, also characterized by the fact that the confidential information protected by said optical film is selected from a group consisting of activation codes, prizes, codes, photographs, logos and/or numbers.

4. The removable optical security film under claim 1 or 2, also characterized by the fact that said security measure has a correct balance between the adhesion force and the removal force (friction) of the security measure to show the information it hides.

5. The removable optical security film under claim 1, also characterized by the fact that the optical elements are selected from a group consisting of holograms, diffraction gradients, optically variable diffraction elements (OVD), dot matrix elements (at any resolution), computer-generated holograms, stereograms, hexelgrams and/or kinegrams.
